(19) United States
(12) Patent Application Publication
(10) Pub. No.: US 2024/0241090 A1
(43) Pub. Date: Jul. 18, 2024

(54) HIGH-RESOLUTION AGE DIFFERENTIATION OF JURASSIC-SOURCED OILS ACROSS THE NORTH ATLANTIC MARGINS

(71) Applicant: EXXONMOBIL TECHNOLOGY AND ENGINEERING COMPANY, Spring, TX (US)

(72) Inventors: Chun ZHU, The Woodlands, TX (US); Mirela DUMITRESCU, Spring, TX (US); Cara L. DAVIS, Houston, TX (US)

(21) Appl. No.: 18/557,718

(22) PCT Filed: Apr. 19, 2022
(86) PCT No.: PCT/US2022/025337

§ 371 Date: Oct. 27, 2023

Related U.S. Application Data
(60) Provisional application No. 63/201,622, filed on May 6, 2021.

(51) Int. Cl. G01N 30/72; E21B 49/08; G01N 30/02; G01N 30/88; G01N 33/28

(52) U.S. Cl. CPC G01N 30/7206; E21B 49/08; G01N 30/88; G01N 33/2823; G01N 33/2835; G01N 2030/025; G01N 2030/8854

(57) ABSTRACT

A method for determining geologic age of hydrocarbon samples, such as oils, using novel age biomarkers may comprise: measuring a concentration or related value of triaromatic dionsteranes (TAD) in a hydrocarbon sample, migrated oil sample, from a North Atlantic conjugate margin (NAM); calculating a TAD index for the hydrocarbon sample based on the concentration or the related value of the TAD; and predicting an age of the hydrocarbon sample based on a correlation between a hydrocarbon age and the TAD index.

### CROSS-REFERENCE TO RELATED APPLICATION

This application is the U.S. National Stage Application of the International Application No. PCT/US2022/025337, entitled “HIGH-RESOLUTION AGE DIFFERENTIATION OF JURASSIC-SOURCED OILS ACROSS THE NORTH ATLANTIC MARGINS,” filed on Apr. 19, 2022, the disclosure of which is hereby incorporated by reference in its entirety, which claims priority to and the benefit of U.S. Provisional Application No. 63/201,622 having a filing date of May 6, 2021, the disclosure of which is incorporated 10) herein by reference in its entirety.

### FIELD OF THE INVENTION

The present disclosure relates to differentiate the geologic age of liquid hydrocarbons (also referred to herein as “oil”). The geologic age of a liquid hydrocarbon, as used herein, refers to the depositional age of the source rock from which the liquid hydrocarbons are derived.

### BACKGROUND

Hydrocarbons accumulated in the subsurface originate from source rocks. Source rocks are often not penetrated in the subsurface because they are found at great depths. Sedimentary organic matter preserved in source rocks is transformed into hydrocarbons with increasing temperature and pressure as it is buried over geologic time. As illustrated in FIG. 1, the hydrocarbons generated in the source rocks, driven by pressure and buoyancy, migrate through carrier beds and accumulate in sealed geological traps that are generally more accessible.

Analogous to physical fossils preserved in sediments, oils contain numerous fossil compounds encoding the generative source rock information. More specifically, when an organism fossilizes, the physical fossil remains in the source rock and chemicals associated with said physical fossil (referred to herein as “chemical fossils”) can leach out. Therefore, just as physical fossils can be linked to geologic age, the chemical fossils can be used to correlate the migrated hydrocarbons to the deeply buried source rock(s), from which the oils are derived.

For example, angiosperm plants have flowers and produce seeds enclosed within a carpel. As the Earth evolved through the Late Cretaceous (100 to 65 million years before present (Ma BP)) epoch through the Paleocene (65 to 55 Ma BP), Eocene (55 to 34 Ma BP), Oligocene (34 to 23 Ma BP), and Miocene (23 to 5 Ma BP) epochs, angiosperms progressively dominated the higher plant community. Therefore, the presence of angiosperm-specific biological markers (known as biomarkers) as chemical fossils of angiosperm in oil can be used to constrain a geologic oil age of Late Cretaceous or younger (i.e., less than (<) 100 Ma BP). More specifically, oleanane (O) is one of the most commonly used angiosperm-specific biomarkers because oleanane survives oil degradation and is easily analyzed using gas spectroscopy-mass spectroscopy (GC-MS). For geologic age constraint, a “concentration” of oleanane (defined as the concentration ratio of oleanane to hopane (H30)) higher than 0.03 has been reported to indicate an age of Cretaceous-Tertiary, or <100 Ma BP as described in Moldowan et al. See Moldowan, J. M., Dahl, J., Huizinga, B. J., Fago, F. J., Hickey, L. J., Peakman, T. M., Taylor, D. W., “The Molecular Fossil Record of Oleanane and Its Relation to Angiosperms,” Science, 265, p. 768 to 777 (1994). FIG. 2 illustrates a chromatogram where oleanane and hopane are labelled. However, an age resolution of <100 Ma BP based on the presence of oleanane is often insufficient to address the age and source rock for hydrocarbon origin in many petroleum basins and makes pinpointing the stratigraphic depth of the corresponding source rocks almost impossible. Other biomarker ratios such as S28/29 (Holba et al., 2001 (ARCO)), BNH/H30 (Grantham and Wakefield, 1988 (Shell)), ETR (Scotchman, 2001 (Equinor); Armstrong et al., 2017 (PSL)), and G/H30 (Scotchman, 2001 (Equinor); Armstrong et al., 2017 (PSL)) have been identified for determining geologic age of oil samples. However, as illustrated in the examples, these age biomarkers do not provide age differentiation within the Jurassic Period (200 to 145 Ma BP) for oils from the North Atlantic conjugated margins (also referred to herein as the NAM). See Holba, A. G., Ellis, L., Dzou, I. L., Hallam, A., Masterson, W. D., Francu, J., Fincannon, A. L., 2001, “Extended tricyclic terpanes as age discriminators between Triassic, Early Jurassic, and Middle-Late Jurassic oils”, In: (abs.) 20th International Meeting on Organic Geochemistry, Sep. 10-14, 2001, Nancy-France, vol. 1, p. 464; Scotchman, I. C., 2001, “Petroleum geochemistry of the Lower and Middle Jurassic in Atlantic margin basins of Ireland and the UK”, In: Shannon, P. M., Haughton, P. D. W. & Corcoran, D. V. (eds) The Petroleum Exploration of Ireland's Offshore Basins. Geological Society, London, Special Publications, 188, p. 31 to p. 60; and Armstrong, H. A., Wagner T., Herringshaw L. G., Farnsworth A. J., Lunt D. J., Harland M., Imber J., Loptson C., and Hadley E. F. L., 2017, “Circulation and precipitation changes controlling black shale deposition in the Late Jurassic Boreal Seaway”, Paleoceanography 31, no. 8: p. 1041 to 1053.

### SUMMARY OF THE INVENTION

The present disclosure relates to determining geologic age of oils using novel age biomarkers.

In one embodiment of the present techniques, a method of the present disclosure may comprise: measuring a concentration or related value of triaromatic dionsteranes (TAD) in a hydrocarbon sample (e.g., migrated oil sample) from a North Atlantic conjugate margin (NAM); calculating a TAD index for the hydrocarbon sample based on the concentration or the related value of the TAD; and predicting an age of the hydrocarbon sample.

In another embodiment of embodiment of the present techniques, a method of the present disclosure may comprise: measuring a concentration or a related value of des-A-hopane (DAH) in a hydrocarbon sample; calculating a DAH index for the hydrocarbon sample within a Kimmeridge epoch and a Tithonian Stage based on the concentration or the related value of the DAH; and predicting an age of the hydrocarbon sample.

Further, a computing device of the present disclosure may comprise: a processor; a memory coupled to the processor; and instructions provided to the memory, wherein the instructions are executable by the processor to perform one or both of the foregoing methods.

### BRIEF DESCRIPTION OF THE DRAWINGS

The following figures are included to illustrate certain aspects of the embodiments, and should not be viewed as exclusive embodiments. The subject matter disclosed is capable of considerable modifications, alterations, combinations, and equivalents in form and function, as will occur to those skilled in the art and having the benefit of this disclosure.

FIG. 1 illustrates the components of a hydrocarbon system in a subsurface region. These components are an effective source rock, thermal maturation, reservoir rocks, migration pathway, seal (cap rock), and trap.

FIG. 2 the maps of North Atlantic conjugated margins for present-day and Jurassic Period.

FIG. 3 is a mass spectrum, proposed structure, and proposed fragmentation pattern for C₂₄ Des-A-Hopane (DAH), a novel geological compound that may be useful in aging oils.

FIG. 4 is a workflow diagram of a nonlimiting example analytical workflow of the present disclosure.

FIG. 5 is a workflow diagram of a nonlimiting example age prediction method of the present disclosure.

FIG. 6 is a partial m/z 245 ion chromatogram for triaromatic dinosteranes (TAD) analysis using gas chromatograph-mass spectroscopy. TAD₁₋₆=six isomers of TAD; C₂₉-4M24E-S=C₂₉ (20S) 4-methyl-24-ethyl triaromatic steranes; C₂₉₋₄M24E-R=C₂₉ (20R) 4-methyl-24-ethyl triaromatic steranes.

FIG. 7 is a partial m/z. 191 ion chromatogram for des-A-hopane (DAH) analysis using gas chromatograph-mass spectroscopy. T21-25=C₂₁₋₂₅ tricyclic terpanes.

FIG. 8 illustrates a plot of the TAD Index values for Lower Jurassic, Middle Jurassic, and Upper Jurassic Epochs in NAM rock samples with known age (the calibration rock set). Lwr J=Lower Jurassic; Mid J=Middle Jurassic; Upper J=Upper Jurassic.

FIG. 9 illustrates a plot of the epoch of the TAD Index values for Tithonian and Kimmeridgian Stages in NAM rock samples with known age (the calibration rock set). Tith=Tithonian; Kimm=Kimmeridgian.

FIG. 10 includes plots of the determined values of published age biomarker ratios S28/29 (Grantham and Wakefield, 1988), BNH/H30 (Scotchman 2001), ETR (Holba et. al., 2001), and G/H30 (Armstrong et al., 2017) in the same NAM rock set against three Jurassic Epochs, specifically the Lower (Lwr), Middle (Mid), and Upper (Upr) Jurassic (J) Periods.

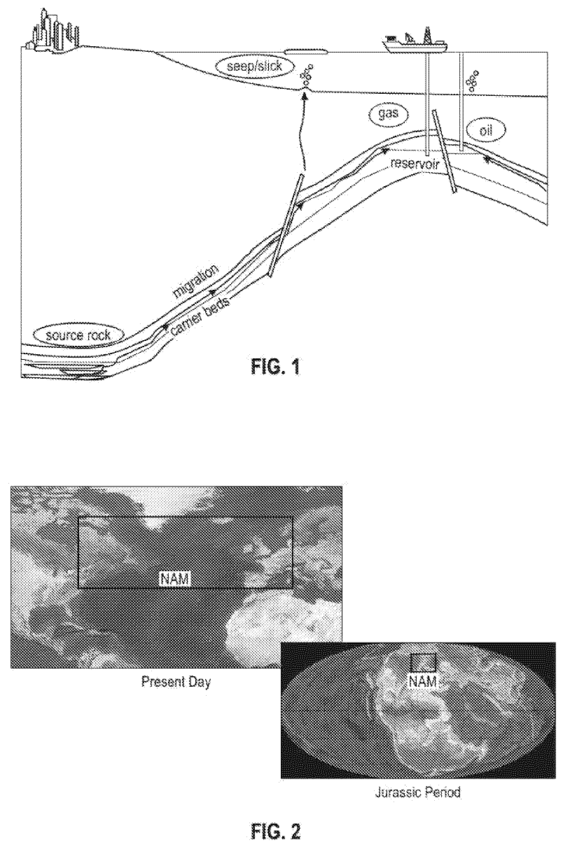

### DETAILED DESCRIPTION

To facilitate a better understanding of the embodiments of the present invention, the examples of preferred or representative embodiments are given within the detailed description. In no way should the following examples be read to limit, or to define, the scope of the invention.

The present disclosure relates to a new geochemical method for age-differentiation of Jurassic marine shale sourced oils across the North Atlantic Margins (NAM). Marine shales deposited during the Jurassic Period (200 to 145 Ma BP) are the major source rocks of crude oils across NAM. There are multiple marine shale sources within the Jurassic Period, and the marine life that deposited to form these marine shale sources varied over time within the Jurassic Period. Marine shale source rocks deposited in different stages of the Jurassic Period can show distinct subsurface distribution (e.g., thickness, depth, and lateral extension), quality, thermal maturity, and generation timing resulting in varying geological and economic risks in hydrocarbon exploration. Therefore, determining the specific age of the Jurassic source rocks can de-risk petroleum system elements in a number of NAM basins. Despite numerous efforts to constrain the ages of marine shale-derived NAM oils over the past several decades, to the best of our knowledge, there are no tools capable of differentiating oil age within the Jurassic Period, particularly Lower Jurassic Epoch versus (vs.) Kimmeridgian Stage vs. Tithonian Stage.

The methods and systems described herein allows for distinguishing oil age (a) within the Jurassic Period (200 to 145 Ma BP) between the Lower Jurassic Epoch (200 to 175 Ma BP) and the Upper Jurassic Epoch (161 to 145 Ma BP) and, further, (b) within the Upper Jurassic Epoch between the Kimmeridgian Stage (155 to 151 Ma BP) and the Tithonian Stage (151 to 145 Ma BP). More specifically, the methods and systems described herein correlate the relative abundance of triaromatic dinosteranes (TADs) in a hydrocarbon sample to differentiate geologic age of the hydrocarbon sample's source between Lower Jurassic or Upper Jurassic Epochs. Further, for samples in the Upper Jurassic Epoch, the relative abundance of des-A-hopane (DAH) in a hydrocarbon sample can be used to correlate the age of the hydrocarbon sample's source to Kimmeridgian or Tithonian Stages. Age may be determined for a single hydrocarbon sample by one or both of the foregoing in the methods and systems of the present disclosure.

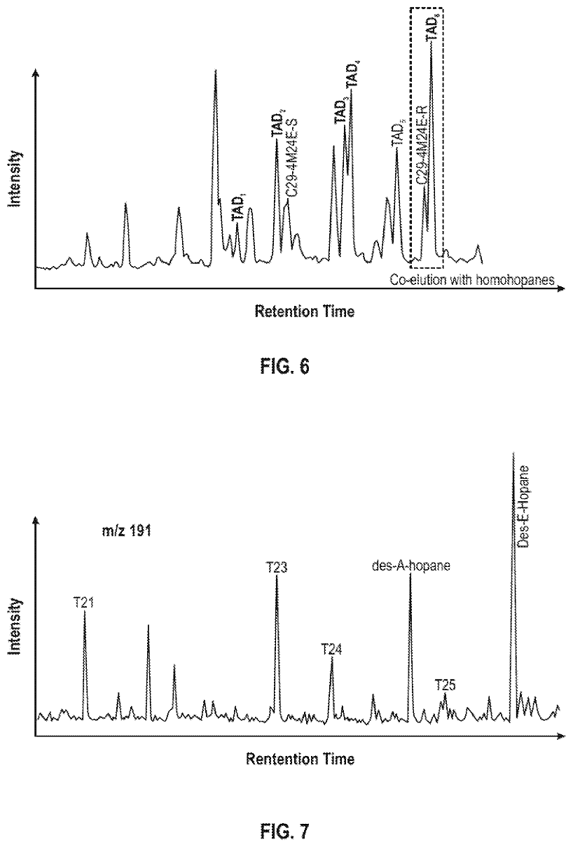

TADs, characterized by three fused aromatic rings, are geological compounds formed in mature sediments via aromatization (Formula 1). There are multiple chiral centers in the TAD molecule, and six (6) TAD isomers can be detected and quantified by gas chromatograph-mass spectroscopy technique (TAD₁₋₆; FIG. 6). The biological precursor of TADs is 4a,23,24-trimethyl-5α-cholest-22E-en-3β-ol, known as dinosterol, a diagnostic membrane lipid biomarker of dinoflagellate organisms (Wolff et al., 1985; Volkman 2003), which first appeared in the Late Triassic/Early Jurassic and diversified through the Mesozoic (Falkowski et al., 2004). See, e.g., Volkman, J. K., 2003, Sterols in microorganisms. Applied Microbiology and Biotechnology 60, p. 495 to 506; Falkowski P. G., Katz M. E., Knoll A. H., Quigg A., Raven J. A, Schofield O., Taylor F. J. R., 2004, The evolution of modern eukaryotic phytoplankton. Science, 305, p. 354 to 360; and Wolff G. A., Lamb N. A., Maxwell J. R., 1985, The origin and fate of 4-methyl steroids-II. Dehydration of stanols and occurrence of C. 4-methyl steranes. Organic Gcochemisty, 10, p. 965 to 974. Therefore, TADs are the chemical fossil of dinoflagellates and detection of TADs from source rocks and crude oils indicates a dinoflagellate input and geologic age specificity.

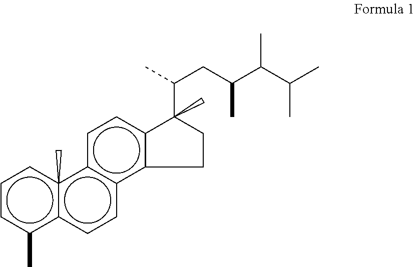

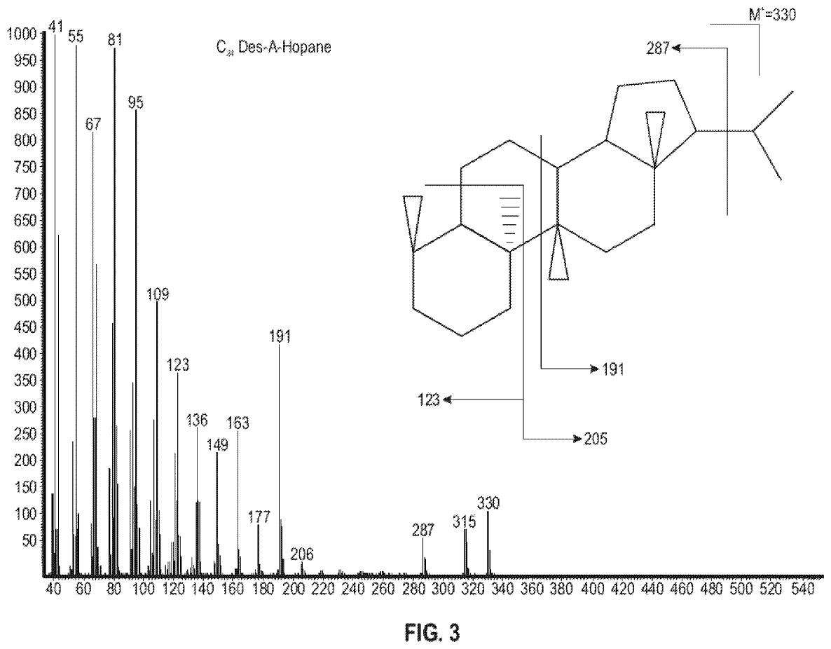

C₂₄ Des-A-Hopane (DAH) is a novel geological compound eluted right before C₂₅ tricyclic terpanes on a gas chromatograph equipped with nonpolar column (e.g., FIG. 7). The proposed structure is provided in FIG. 3 based on the full mass spectrum and fragmentation pattern of gas chromatograph-mass spectroscopy analysis in this study. This is interpreted as a tetracyclic terpane bearing an isopropyl moiety, likely a degradation product of C₃₀ hapanol/hapenol through A ring cleavage during diagenesis.

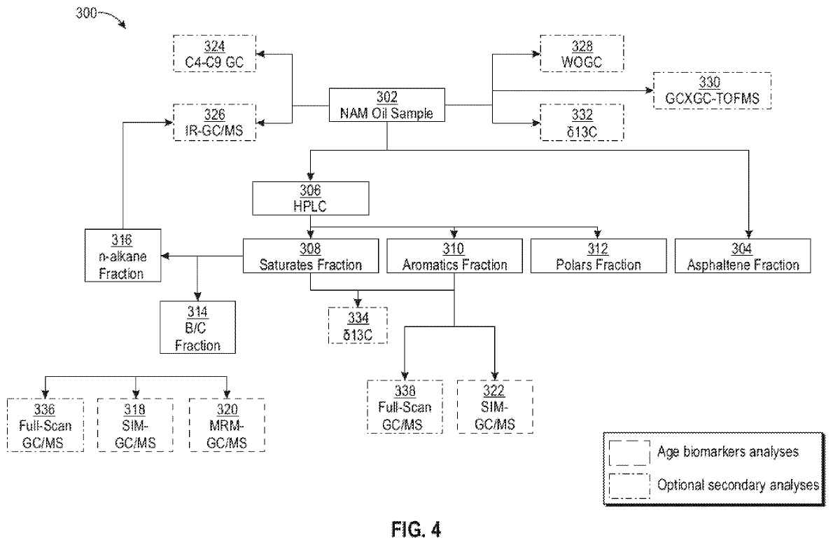

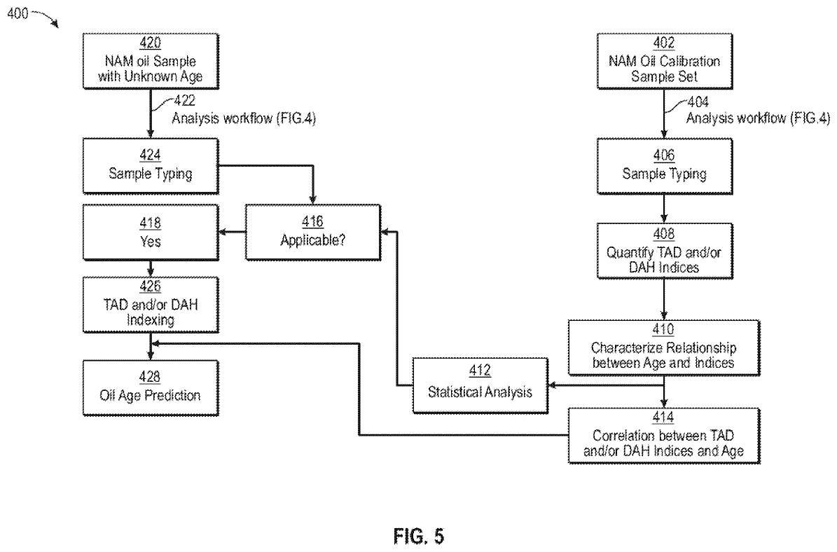

Because crude oils are a complex mixture of natural products, oil samples need to be fractionated to remove compounds that interfere the detection of age-diagnostic biomarkers. FIG. 4 is a nonlimiting example of a sample analytical flowchart (300) for age-diagnostic biomarker analysis of a NAM oil sample 302. First, the NAM oil sample 302 is separated in to a plurality of fractions, which as illustrated may include an asphaltene fraction 304, a saturated fraction 308, an aromatic fraction 310, and a polar fraction 312. For example, the asphaltenes may be precipitated from the NAM oil sample 302 using n-pentane. The remaining pentane-soluble portion of the NAM oil sample 302 may be separated into the saturated fraction 308, the aromatic fraction 308, and the polar fraction 310 using high-pressure liquid chromatography (HPLC) 306. The saturated fraction may be further purified using a molecular sieve (e.g., zeolite or synthetic zeolite which contains silica (Si) and alumina (Al) with the ratio of silica greater than the alumina, such as ZSM-5) to isolate branched and cyclic alkanes (B/C fraction) 314 by removing the normal alkanes 316. The B/C fraction 314 may then be analyzed by selective ion monitoring-gas chromatography/mass spectroscopy (SIM-GC/MS) 318 and metastable reaction monitoring-gas chromatography/mass spectroscopy tandem mass spectroscopy (MRM-GC/MSMS) 320. The aromatic fraction from the HPLC separation may also analyzed by SIM-GC/MS 322.

The results from the SIM-GC/MS 318, the MRM-GC/MSMS 320, and the SIM-GC/MS 322 may then be analyzed to determine the relative abundances of TAD and/or DAH and ultimately the geologic age of the source of the NAM oil sample 302. This portion of the methods and systems is described further herein.

Generally. SIM-GC/MS and MRM-GC/MSMS techniques have higher resolution than a full-scan GC/MS, and both techniques may provide a more accurate measurement of TAD and/or DAH concentrations.

In the methods and systems described herein, one or more other secondary analyses may be performed on the whole hydrocarbon sample and/or fractions thereof to ascertain a level of biodegradation, a level of contamination, sample maturity, sulfur content, American Petroleum Institute (API) gravity, and other characteristics of the hydrocarbon sample, which are useful in evaluating potential issues with sample quality that could impact age determination. One skilled in the art will recognize suitable analysis techniques for measuring such characteristics. Examples of analysis techniques include, but are not limited to, GCxGC-TOFMS (time of flight mass spectroscopy) 330, whole oil gas chromatography (WOGC) 328, ¹³C isotopic composition (δ¹³C) 334 and 332, full-scan GCMS 336 and 338, infrared (IR)-GC/MS 326, and Ca to C₁₉ GC 324.

Generally, GCxGC-TOFMS is used to confirm the chemical structures of age-diagnostic biomarkers preserved in a crude oil sample. WOGC helps assess the degree of biodegradation and artificial contamination that may impact age biomarker distribution. δ¹³C relates to bulk isotopic characteristic of the hydrocarbon sample. Any saturated and aromatic fractions are analyzed by δ¹³C for the fraction-specific isotopic characteristic of the hydrocarbon sample. The aromatic and the B/C fractions are analyzed by full-scan GC/MS to confirm chemical structures of age-diagnostic biomarkers of the hydrocarbon sample. The n-alkanes separated from the saturate fraction are analyzed by isotopic ratio GCMS (IR-GCMS), which relates to isotopic characteristic of n-alkanes of the hydrocarbon sample).

Any of the foregoing analytical techniques may be replaced or augmented with comparable techniques known to one of skill in the art.

FIG. 5 is a nonlimiting example flow diagram of a method (400) for age differentiation within the Jurassic Period of NAM sourced samples. First, a calibration sample set 402 of quality oil and source rock samples with known specific age during Jurassic Period are collected.

The calibration samples 402 may be analyzed by the analytical workflow 300 shown in FIG. 4. From the SIM-GCMS and MRM-GCMS results of the B/C fraction and the SIM-GCMS results of the aromatic fraction, the molecular composition of calibration samples may be geochemical characterized (or source typed 406) in terms of source facies, maturity, and alteration. The other characteristics of the samples may be derived from secondary analyses that may be used (and were used in this example) to confirm the source type by maturity, source facies, and alteration of the oil samples.

Subsequently, the distribution of TAD in these calibration samples may be used to quantify 408 a TAD index (Eq. 1) from the SIM-GCMS of the saturated or B/C fraction. Likewise, the DAH index (Eq. 2) may be quantified from the SIM-GCMS results of the aromatic fraction. This indices may be a relative concentration to another compound(s) in the sample. For example, Equation 1 may be used for TAD Index, and Equation 2 may be used for the DAH Index. Other quantifications may be used to characterize each of the indices. For example, the TAD Index may be the TAD concentration relative to C₂₉ (20S) 4-methyl-24-ethyl triaromatic steranes (C29-4M24E-S) or a different compound. In another example, the TAD Index may use fewer than TAD₁-5 isomers in characterizing the TAD concentration. In another example, the DAH Index may be relative to C₂₅ tricyclic terpanes, a sum of multiple tricyclic terpanes, or a different compound.

$$TAD Index = ( \sum [ {TAD}_{1 - 5} ] ) / [ C 29 - 4 M 24 E - S ] \qquad Equation 1$$

where TAD₁-5 is the five of the TAD isomers and C29-4M24E-S is the C₂₉ (20S) 4-methyl-24-ethyl triaromatic steranes

$$DAH Index = ( [ DAH ] 2 ) / ( \sum [ T 25 ] ) \qquad Equation 2$$

where T25 are the two isomers of C₂₅ tricyclic terpane-S and C₂₅ tricyclic terpane-R

As used herein, when referring to a relative concentration of a biomarker and when using bracket “[ ]” concentration or chromatographic peak intensity designations in a formula, the value used may be a true concentration or a value related to concentration. For example, the intensity of a peak in a GC/MS chromatogram relates to the concentration of the corresponding composition. Accordingly, the peak intensity can be used as a value related to the concentration in the formulas and methods described herein.

FIG. 6 is a partial m/z 245 ion chromatograph showing the elution of several TAD isomers and the C29-4M24E-S. FIG. 7 is a partial m/z 191 ion chromatograph showing the elution of DAH and T25.

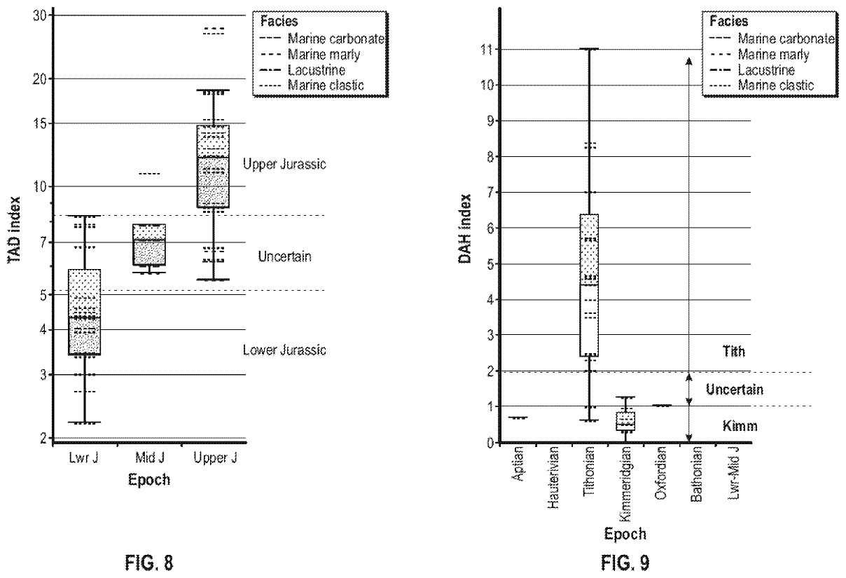

A correlation between each of the TAD Index values and/or the DAH Index values and the age of calibration samples may then be characterized 410. FIG. 8 illustrates a relationship of TAD Index with the age of the calibration rock samples separated into Lower Jurassic, Middle Jurassic, and Upper Jurassic Epochs. FIG. 9 illustrates a relationship of the DAH Index with the age of the calibration rock samples separated into several Stages including Tithonian and Kimmeridgian.

Referring again to FIG. 5, the sample characteristics 412 that should be met for the samples to be properly correlated with TAD Index and/or the DAH Index are determined by statistical analysis. For example, the calibration samples that can be effectively differentiated between Lower and Upper Jurassic Epochs using the TAD Index and those that cannot be effectively differentiated are compared to determine what sample conditions or properties are generally required (or preferred) to yield an effective differentiation. Examples of some sample properties include maturity level, source facies, degree and/or type of sample alterations, and the like.

The result of the calibration portion of the method is (1) the sample characteristics 412 that should be met to apply the TAD Index and/or the DAH Index for sample aging and (2) the relationship 414 (or correlation) between the TAD Index and/or the DAH Index and sample age. Using Equations 1 and 2 for the TAD Index and the DAH Index, respectively, it has been determined that an estimated age of a sample having a TAD Index of 5 or less is Lower Jurassic Epoch, an estimated age of a sample having a DAH Index of 2 or more is Tithonian Stage, an estimated age of a sample having a DAH Index of 1 or less is Kimmeridgian Stage. When using other equations to describe the TAD Index and/or the DAH Index, the foregoing values may change.

Referring again to FIG. 5, a method for aging a NAM oil of unknown age 420 may include performing 422 an analysis workflow according to FIG. 4. From the results of the analysis workflow, the sample typing 424 (e.g., maturity, source facies, and/or alteration of the oil samples) may be performed. The sample type of the NAM oil of unknown age 420 may be compared 416 to the sample characteristics 412 that should be met for the samples to be properly correlated with TAD Index and/or the DAH Index. If the NAM oil of unknown age 420 meets 418 said sample characteristics 412, the TAD, DAH, and other compound concentrations may be determined based on the results from the analysis workflow. Then, the TAD Index and/or the DAH Index for the NAM oil of unknown age 420 may be determined 426 and compared to the correlation 414 between the TAD Index and/or the DAH Index and age to arrive at an estimated oil age 428 for the NAM oil of unknown age 420.

Various aspects of the systems and methods described herein utilize computer systems. Such systems and methods can include a non-transitory computer readable medium containing instructions that, when implemented, cause one or more processors to carry out the methods described herein.

“Computer-readable medium” or “non-transitory, computer-readable medium,” as used herein, refers to any non-transitory storage and/or transmission medium that participates in providing instructions to a processor for execution. Such a medium may include, but is not limited to, non-volatile media and volatile media. Non-volatile media includes, for example, NVRAM, or magnetic or optical disks. Volatile media includes dynamic memory, such as main memory. Common forms of computer-readable media include, for example, a floppy disk, a flexible disk, a hard disk, an array of hard disks, a magnetic tape, or any other magnetic medium, magneto-optical medium, a CD-ROM, a holographic medium, any other optical medium, a RAM, a PROM, and EPROM, a FLASH-EPROM, a solid state medium like a memory card, any other memory chip or cartridge, or any other tangible medium from which a computer can read data or instructions. When the computer-readable media is configured as a database, it is to be understood that the database may be any type of database, such as relational, hierarchical, object-oriented, and/or the like. Accordingly, exemplary embodiments of the present systems and methods may be considered to include a tangible storage medium or tangible distribution medium and prior art-recognized equivalents and successor media, in which the software implementations embodying the present techniques are stored.

The methods described herein can be performed using computing devices or processor-based devices that include a processor; a memory coupled to the processor; and instructions provided to the memory, wherein the instructions are executable by the processor to perform the methods described herein. The instructions can be a portion of code on a non-transitory computer readable medium. Any suitable processor-based device may be utilized for implementing all or a portion of embodiments of the present techniques, including without limitation personal computers, networks of personal computers, laptop computers, computer workstations, mobile devices, multi-processor servers or workstations with (or without) shared memory, high performance computers, and the like. Moreover, embodiments may be implemented on application specific integrated circuits (ASICs) or very large scale integrated (VLSI) circuits.

### Applications of Oil Aging

The methods and systems described herein may be useful in distinguishing oil age (a) within the Jurassic Period between the Lower Jurassic Epoch (200 to 175 Ma BP) and the Upper Jurassic Epoch (161 to 145 Ma BP) and, further, (b) within the Upper Jurassic Epoch between the Kimmeridgian Stage (155 to 151 Ma BP) and the Tithonian Stage (151 to 145 Ma BP). As such, the methods and systems described herein may provide a better understanding of the origin of oils, oil stains, or oil seeps, which allows one to de-risk hydrocarbon charge (the volume of hydrocarbons expected to be delivered to a trap) and better differentiates exploration opportunities.

In a first nonlimiting example, the specific age of oils, oil stains, or oil seeps ties the samples to a specific stratigraphic interval (or source rock) where hydrocarbons are derived. As a result, hydrocarbon migration pathways from the source to trap can be illustrated. The migration pathway may determine whether source rocks can effectively charge the location (e.g., a trap, a basin, or the like) from which the hydrocarbon sample was retained to form economic hydrocarbon accumulation. Therefore, in some instances, once the source rock and migration pathway has been determined, a wellbore may be drilled into the location (e.g., a trap, a basin, or the like) from which the hydrocarbon sample was obtained. When referring to location here, the exact location is not implied by rather a general location of the trap, the seep, the basin, or the like that contains oil from the same source rock as the hydrocarbon sample.

In another nonlimiting example, the hydrocarbon generation timing can be calculated based on the specific oil age and the thermal history of the basin. The resultant generation timing is further compared with the timing of trap emplacement. If the trap is deposited later than peak generation of hydrocarbons, the source rock is unlikely to charge the trap and it is considered highly risky to form economic hydrocarbon accumulation.

Other applications of aging and the benefits of better resolved aging per the methods and systems described herein will be apparent to those skilled in the art.

### Example Embodiments

A first nonlimiting example embodiment of the present disclosure is a method comprising: measuring a concentration or related value of triaromatic dionsteranes (TAD) in a hydrocarbon sample (e.g., migrated oil sample) from a North Atlantic conjugate margin (NAM); calculating a TAD index for the hydrocarbon sample based on the concentration or the related value of the TAD; and predicting an age of the hydrocarbon sample based on a correlation between a hydrocarbon age and the TAD index. The first nonlimiting example embodiment may further include one or more of: Element 1: wherein the measuring of the concentration or the related value of the TAD comprises: separating the hydrocarbon sample into separated fractions that include an aromatic fraction and a saturated fraction; isolating a branched and cyclic alkanes fraction from the saturated fraction if the yield of saturated fraction is sufficient (e.g., greater than (>) 20 milligram (mg)); performing selective ion monitoring-gas chromatography/mass spectroscopy and metastable reaction monitoring-gas chromatography/mass spectroscopy tandem mass spectroscopy on the saturated fraction and/or the branched and cyclic alkanes fraction; and performing selective ion monitoring-gas chromatography/mass spectroscopy on the aromatic fraction; Element 2: the method further comprising: performing one or more analyses selected from the group consisting of time of flight mass spectroscopy, whole oil gas chromatography, ¹³C isotopic composition, full-scan gas chromatography/mass spectroscopy, infrared-gas chromatography/mass spectroscopy, and C₄ to C₁₉ gas chromatography on the hydrocarbon sample or a separated fraction thereof; Element 3: the method further comprising: identifying a source rock (e.g., a generative source rock) of the hydrocarbon sample based on the age of the hydrocarbon sample; and estimating and/or constraining a migration pathway for a hydrocarbon source of the hydrocarbon sample based on the source rock and a location from which the oil sample was obtained; Element 4: the method further comprising: performing a pre-drill risk evaluation of a hydrocarbon charge for a prospect based on the age of the hydrocarbon sample and a thermal history of the location from which the hydrocarbon sample was obtained; Element 5: the method further comprising: drilling a wellbore into a location from which the hydrocarbon sample was obtained; Element 6: the method further comprising: producing hydrocarbon from a location from which the hydrocarbon sample was obtained. Examples of combinations include, but are not limited to, Element 1 in combination with one or more of Elements 2 to 6; Element 2 in combination with one or more of Elements 3 to 6; Element 3 in combination with one or more of Elements 4 to 6; and two or more of Elements 4 to 6 in combination.

A second nonlimiting example embodiment is a computing device comprising: a processor; a memory coupled to the processor; and instructions provided to the memory, wherein the instructions are executable by the processor to perform the method of the first nonlimiting example embodiment.

A third nonlimiting example embodiment is a method comprising: measuring a concentration or a related value of des-A-hopane (DAH) in a hydrocarbon sample; calculating a DAH index for the hydrocarbon sample within a Kimmeridge epoch and a Tithonian Stage based on the concentration or the related value of the DAH; and predicting an age of the hydrocarbon sample based on a relationship between hydrocarbon age and the DAH index. The second nonlimiting example embodiment may further include one or more of: Element 7: wherein the measuring of the concentration or the related value of the DAH comprises: separating the hydrocarbon sample into separated fractions that include an aromatic fraction and a saturated fraction; performing selective ion monitoring-gas chromatography/mass spectroscopy and metastable reaction monitoring-gas chromatography/mass spectroscopy tandem mass spectroscopy on the aromatic fraction; and performing selective ion monitoring-gas chromatography/mass spectroscopy on the aromatic fraction; Element 8: the method further comprising: performing one or more analyses selected from the group consisting of time of flight mass spectroscopy, whole oil gas chromatography, ¹³C isotopic composition, full-scan gas chromatography/mass spectroscopy, infrared-gas chromatography/mass spectroscopy, and C₄ to C₁₉ gas chromatography on the hydrocarbon sample or a separated fraction thereof; Element 9: the method further comprising: identifying a source rock of the hydrocarbon sample based on the age of the hydrocarbon sample; and estimating and/or constraining a migration pathway for a hydrocarbon source of the hydrocarbon sample based on the source rock and a location from which the oil sample was obtained; Element 10: the method further comprising: performing a pre-drill risk evaluation of hydrocarbon charge for a prospect based on the age of the hydrocarbon sample and a thermal history of the location from which the hydrocarbon sample was obtained; Element 11: the method further comprising: drilling a wellbore into a location from which the hydrocarbon sample was obtained; Element 12: the method further comprising: producing hydrocarbon from a location from which the hydrocarbon sample was obtained. Examples of combinations include, but are not limited to, Element 7 in combination with one or more of Elements 8 to 12; Element 8 in combination with one or more of Elements 9 to 12; Element 9 in combination with one or more of Elements 10 to 12; and two or more of Elements 10 to 12 in combination.

A fourth nonlimiting example embodiment is a computing device comprising: a processor; a memory coupled to the processor; and instructions provided to the memory, wherein the instructions are executable by the processor to perform the method of the third nonlimiting example embodiment.

Unless otherwise indicated, all numbers expressing quantities of ingredients, properties such as molecular weight, reaction conditions, and so forth used in the present specification and associated claims are to be understood as being modified in all instances by the term “about.” Accordingly, unless indicated to the contrary, the numerical parameters set forth in the following specification and attached claims are approximations that may vary depending upon the desired properties sought to be obtained by the embodiments of the present invention. At the very least, and not as an attempt to limit the application of the doctrine of equivalents to the scope of the claim, each numerical parameter should at least be construed in light of the number of reported significant digits and by applying ordinary rounding techniques.

One or more illustrative embodiments incorporating the invention embodiments disclosed herein are presented herein. Not all features of a physical implementation are described or shown in this application for the sake of clarity. It is understood that in the development of a physical embodiment incorporating the embodiments of the present invention, numerous implementation-specific decisions are made to achieve the developer's goals, such as compliance with system-related, business-related, government-related and other constraints, which vary by implementation and from time to time. While a developer's efforts might be time-consuming, such efforts would be, nevertheless, a routine undertaking for those of ordinary skill in the art and having benefit of this disclosure.

While compositions and methods are described herein in terms of “comprising” various components or steps, the compositions and methods can also “consist essentially of” or “consist of” the various components and steps.

### Examples

A set of NAM rock samples of known age (determined by biostratigraphy) were used as a calibration sample set and analyzed according to the method described in FIG. 4 including the SIM-GC/MS 318, the MRM-GC/MSMS 320, and the SIM-GC/MS 322 analyses. In this specific example, the calibration sample set included over 51 quality rock samples with known specific age during the Jurassic Period (including during the Lower Jurassic Epoch and the Upper Jurassic Epoch and within the Upper Jurassic Epoch during the Kimmeridgian Stage and the Tithonian Stage) from petroleum basins across the NAM.

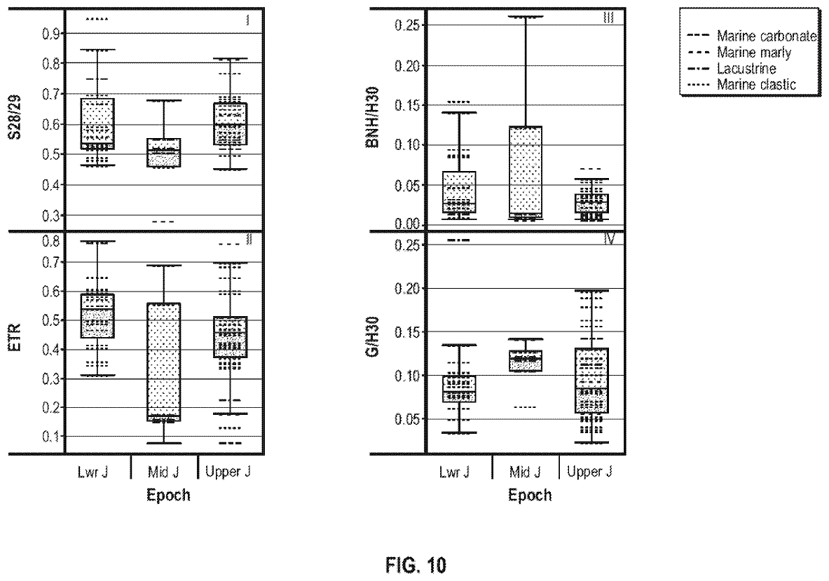

Several published age biomarker ratios include S28/29 (Grantham and Wakefield, 1988), BNH/H30 (Scotchman, 2001), ETR (Holba et al., 2001), and G/H30 (Armstrong et al., 2017) were tested for their accuracy of age differentiation between Upper and Lower Jurassic Epochs using NAM rock samples with known age. FIG. 10 includes plots for each of the S28/29, BNH/H30, ETR, and G/H30 values against the Lower, Middle, and Upper Jurassic Epochs. See Grantham, P. J., Wakefield, L. L., 1988, “Variations in the sterane carbon number distributions of marine source rock derived crude oils through geological time”, Organic Geochemistry 12, p. 61 to 73. The plots of FIG. 10 illustrates that these published age biomarkers fail to further differentiate the specific age within Jurassic Period for the NAM rocks and therefore they are not applicable to age differentiation within the Jurassic Period (200-145 Ma BP) for the NAM oils. To the best of our knowledge, there is no published biomarker approach capable of distinguishing oil age between Kimmeridgian Stage and Tithonian Stage.

In contrast, the TAD Index and DAH Index described herein were determined for the samples and a correlation to age was determined according to FIG. 5. FIGS. 8 and 9 are the correlation between age and TAD Index and the correlation between age and DAH Index, respectively, that were determined using the same rock samples as the FIG. 10 correlations.

This illustrates the TAD Index and the DAH Index as unique methods for aging NAM oil samples within the Jurassic Period.

The following example embodiments of the invention are also disclosed.

Embodiment 1. A method comprising: measuring a concentration or related value of triaromatic dionsteranes (TAD) in a hydrocarbon sample from a North Atlantic conjugate margin; calculating a TAD index for the hydrocarbon sample based on the concentration or the related value of the TAD; and predicting an age of the hydrocarbon sample based on a correlation between a hydrocarbon age and the TAD index.

Embodiment 2. The method of Embodiment 1, wherein the measuring of the concentration or the related value of the TAD comprises: separating the hydrocarbon sample into separated fractions that include an aromatic fraction and a saturated fraction; isolating a branched and cyclic alkanes fraction from the saturated fraction if the yield of saturated fraction is sufficient; performing selective ion monitoring-gas chromatography/mass spectroscopy and metastable reaction monitoring-gas chromatography/mass spectroscopy tandem mass spectroscopy on the saturated fraction and/or the branched and cyclic alkanes fraction; and performing selective ion monitoring-gas chromatography/mass spectroscopy on the aromatic fraction.

Embodiment 3. The method of Embodiment 1 further comprising: performing one or more analyses selected from the group consisting of time of flight mass spectroscopy, whole oil gas chromatography, 13C isotopic composition, full-scan gas chromatography/mass spectroscopy, infrared-gas chromatography/mass spectroscopy, and C4 to C19 gas chromatography on the hydrocarbon sample or a separated fraction thereof.

Embodiment 4. The method of Embodiment 1 further comprising: identifying a source rock of the hydrocarbon sample based on the age of the hydrocarbon sample; and estimating and/or constraining a migration pathway for a hydrocarbon source of the hydrocarbon sample based on the source rock and a location from which the oil sample was obtained.

Embodiment 5. The method of Embodiment 1 further comprising: performing a pre-drill risk evaluation of a hydrocarbon charge for a prospect based on the age of the hydrocarbon sample and a thermal history of the location from which the hydrocarbon sample was obtained.

Embodiment 6. The method of Embodiment 1 further comprising: drilling a wellbore into a location from which the hydrocarbon sample was obtained.

Embodiment 7. The method of Embodiment 1 further comprising: producing hydrocarbon from a location from which the hydrocarbon sample was obtained.

Embodiment 8. A method comprising: measuring a concentration or a related value of des-A-hopane (DAH) in a hydrocarbon sample; calculating a DAH index for the hydrocarbon sample within a Kimmeridge epoch and a Tithonian Stage the based on the concentration or the related value of the DAH; and predicting an age of the hydrocarbon sample based on a relationship between hydrocarbon age and the DAH index.

Embodiment 9. The method of Embodiment 8, wherein the measuring of the concentration or the related value of the DAH comprises: separating the hydrocarbon sample into separated fractions that include an aromatic fraction and a saturated fraction; performing selective ion monitoring-gas chromatography/mass spectroscopy and metastable reaction monitoring-gas chromatography/mass spectroscopy tandem mass spectroscopy on the aromatic fraction; and performing selective ion monitoring-gas chromatography/mass spectroscopy on the aromatic fraction.

Embodiment 10. The method of Embodiment 8 further comprising: performing one or more analyses selected from the group consisting of time of flight mass spectroscopy, whole oil gas chromatography, 13C isotopic composition, full-scan gas chromatography/mass spectroscopy, infrared-gas chromatography/mass spectroscopy, and C4 to C19 gas chromatography on the hydrocarbon sample or a separated fraction thereof.

Embodiment 11. The method of Embodiment 8 further comprising: identifying a source rock of the hydrocarbon sample based on the age of the hydrocarbon sample; and estimating and/or constraining a migration pathway for a hydrocarbon source of the hydrocarbon sample based on the source rock and a location from which the oil sample was obtained.

Embodiment 12. The method of Embodiment 8 further comprising: performing a pre-drill risk evaluation of hydrocarbon charge for a prospect based on the age of the hydrocarbon sample and a thermal history of the location from which the hydrocarbon sample was obtained.

Embodiment 13. The method of Embodiment 8 further comprising: drilling a wellbore into a location from which the hydrocarbon sample was obtained.

Embodiment 14. The method of Embodiment 8 further comprising: producing hydrocarbon from a location from which the hydrocarbon sample was obtained.

Embodiment 15. A computing device comprising: a processor; a memory coupled to the processor; and instructions provided to the memory, wherein the instructions are executable by the processor to perform the method of any preceding Embodiment 1 to Embodiment 15.

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope and spirit of the present invention. The invention illustratively disclosed herein suitably may be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces.

1. A method comprising:
measuring a concentration or related value of triaromatic dionsteranes (TAD) in a hydrocarbon sample from a North Atlantic conjugate margin;
calculating a TAD index for the hydrocarbon sample based on the concentration or the related value of the TAD; and
predicting an age of the hydrocarbon sample based on a correlation between a hydrocarbon age and the TAD index.

2. The method of claim 1, wherein the measuring of the concentration or the related value of the TAD comprises:
separating the hydrocarbon sample into separated fractions that include an aromatic fraction and a saturated fraction;
isolating a branched and cyclic alkanes fraction from the saturated fraction if the yield of saturated fraction is sufficient;
performing selective ion monitoring-gas chromatography/mass spectroscopy and metastable reaction monitoring-gas chromatography/mass spectroscopy tandem mass spectroscopy on the saturated fraction and/or the branched and cyclic alkanes fraction; and
performing selective ion monitoring-gas chromatography/mass spectroscopy on the aromatic fraction.

3. The method of claim 1, comprising:
performing one or more analyses selected from the group consisting of time of flight mass spectroscopy, whole oil gas chromatography, ¹³C isotopic composition, full-scan gas chromatography/mass spectroscopy, infrared-gas chromatography/mass spectroscopy, and C₄ to C₁₉ gas chromatography on the hydrocarbon sample or a separated fraction thereof.

4. The method of claim 1, comprising:
identifying a source rock of the hydrocarbon sample based on the age of the hydrocarbon sample; and
estimating and/or constraining a migration pathway for a hydrocarbon source of the hydrocarbon sample based on the source rock and a location from which the oil sample was obtained.

5. The method of claim 1, comprising:
performing a pre-drill risk evaluation of a hydrocarbon charge for a prospect based on the age of the hydrocarbon sample and a thermal history of the location from which the hydrocarbon sample was obtained.

6. The method of claim 1, comprising:
drilling a wellbore into a location from which the hydrocarbon sample was obtained.

7. The method of claim 1, comprising:
producing hydrocarbon from a location from which the hydrocarbon sample was obtained.

8. A method comprising:
measuring a concentration or a related value of des-A-hopane (DAH) in a hydrocarbon sample;
calculating a DAH index for the hydrocarbon sample within a Kimmeridge epoch and a Tithonian Stage the based on the concentration or the related value of the DAH; and
predicting an age of the hydrocarbon sample based on a relationship between hydrocarbon age and the DAH index.

9. The method of claim 8, wherein the measuring of the concentration or the related value of the DAH comprises:
separating the hydrocarbon sample into separated fractions that include an aromatic fraction and a saturated fraction;
performing selective ion monitoring-gas chromatography/mass spectroscopy and metastable reaction monitoring-gas chromatography/mass spectroscopy tandem mass spectroscopy on the aromatic fraction; and
performing selective ion monitoring-gas chromatography/mass spectroscopy on the aromatic fraction.

10. The method of claim 8, comprising:
performing one or more analyses selected from the group consisting of time of flight mass spectroscopy, whole oil gas chromatography, ¹³C isotopic composition, full-scan gas chromatography/mass spectroscopy, infrared-gas chromatography/mass spectroscopy, and C₄ to C₁₉ gas chromatography on the hydrocarbon sample or a separated fraction thereof.

11. The method of claim 8, comprising:
identifying a source rock of the hydrocarbon sample based on the age of the hydrocarbon sample; and
estimating and/or constraining a migration pathway for a hydrocarbon source of the hydrocarbon sample based on the source rock and a location from which the oil sample was obtained.

12. The method of claim 8, comprising:
performing a pre-drill risk evaluation of hydrocarbon charge for a prospect based on the age of the hydrocarbon sample and a thermal history of the location from which the hydrocarbon sample was obtained.

13. The method of claim 8, comprising:
drilling a wellbore into a location from which the hydrocarbon sample was obtained.

14. The method of claim 8, comprising:
producing hydrocarbon from a location from which the hydrocarbon sample was obtained.

15. A computing device comprising:
a processor;
a memory coupled to the processor; and
instructions provided to the memory, wherein the instructions are executable by the processor to perform the method of any preceding claim.
